(19) United States
(12) Patent Application Publication
(10) Pub. No.: US 2002/0139719 A1
(43) Pub. Date: Oct. 3, 2002

(54) Removal of thiophenic sulfur from gasoline by membrane separation process

(76) Inventors: Bhupender S. Minhas, Bel Air, MD (US); Robert J. Saxton, Pleasanton, CA (US); Ingo Pinnau, Palo Alto, CA (US); Zhenjie He, Fremont, CA (US); Johannes G. Wijmans, Menlo Park, CA (US)

Correspondence Address: EXXONMOBIL RESEARCH AND ENGINEERING COMPANY, P.O. BOX 900, 1545 ROUTE 22 EAST, ANNANDALE, NJ, 08801-0900, US

(21) Appl. No.: 10/021,802

(22) Filed: Dec. 12, 2001

Related U.S. Application Data
(60) Provisional application No. 60/258,485, filed on Dec. 28, 2000.

(51) Int. Cl. C07C 5/00

(52) U.S. Cl. 208/250

(57) ABSTRACT

Methods for the separation of sulfur compounds from a liquid hydrocarbon mixture using a hydrophilic, non-ionic membrane are provided. The membrane can also be composed of water-soluble material. Preferred membranes include polyvinylpyrrolidone and cellulose triacetate membranes. The liquid hydrocarbon mixture may be light cracked naphtha.

### CROSS REFERENCE TO RELATED APPLICATIONS

This application claims benefit of Provisional U.S. Application Serial No. 60/258,485 filed Dec. 28, 2000.

### FIELD OF THE INVENTION

The present invention relates to the separation of sulfur compounds from hydrocarbon mixtures using a non-ionic membrane. BACKGROUND OF THE INVENTION

Sulfur compounds are impurities in gasoline that compromise vehicle emission controls by poisoning the catalytic converter. In an effort to further decrease emissions, the U.S. government has recently proposed a nationwide reduction of sulfur in gasoline from current levels at 300-1000 ppm to an average of 30 ppm (Federal Register, 64(92), May 13, 1999). Gasoline producers, both domestic and foreign, selling fuel in the U.S. would be expected to comply by the year 2004.

Presently, the conventional process for reducing sulfur content in gasoline involves hydrotreating in which sulfur compounds are converted to volatile hydrogen sulfide and other organics. This energy intensive process, requiring elevated temperature and pressure, is expensive for obtaining the proposed lowered sulfur levels. Alternative processes with more efficient sulfur-reducing technology are needed to maintain progress toward cleaner burning fuels.

The use of membrane separation technology, in which select compounds or types or compounds can be separated from an organic mixture by permeation through a membrane, has been reasonably well developed. Separation processes that incorporate membranes present an attractive option for large scale treatment of petroleum fractions because of their inherent simplicity, versatility, and low energy consumption.

Typically, membrane separation processes rely on the affinity of a specific compound or class of compounds for the membrane. In this way, the components of a mixture with specific affinity for the membrane will selectively sorb onto the membrane. The sorbed compounds diffuse, or permeate, through the membrane and are removed on the opposite side. Continual withdrawal of permeated compounds from the membrane maintains the driving force for the separation process. Removal of permeated compounds is usually achieved by pervaporation or perstraction methods. Pervaporation employs a vacuum on the permeate side of the membrane, removing the permeated compounds in gaseous form, while perstraction employs a liquid sweep stream, continually washing away permeate.

The chemical properties of the membrane dictate the type of compounds that have affinity for it. Some types of membranes are composed of charged chemical groups and are therefore considered ionic. In contrast, non-ionic membranes are made from those materials lacking charged chemical groups. Chemical affinity is usually governed by the hydrophilic or hydrophobic nature of the membrane material. Hydrophilic membranes have affinity for water or other polar compounds. Hydrophilic membranes include both ionic and non-ionic membranes. However, the non-ionic membranes generally contain polar chemical groups such as hydroxyl, carboxyl, sulfonyl, carbonyl, or amine groups. Examples of hydrophilic non-ionic membranes include polyvinylalcohol (PVA), cellulose acetates, and polyvinylamine. Hydrophobic membranes, on the other hand, have little affinity for water or polar compounds and generally lack or contain a small proportion of charged or polar chemical groups. Examples of hydrophobic membranes include polyethylene and polystyrene.

A wide variety of non-ionic membranes have been used in separation processes. U.S. Pat. Nos. 5,905,182, 5,019,666, 4,997,906, 4,944,880, 4,532,029, 4,802,987, 4,962,271, 5,288,712, 5,635,055, 3,556,991, 3,043,891, and 2,947,687 demonstrate the separation of aromatics from hydrocarbon mixtures using a variety of non-ionic membrane materials. Non-ionic membranes have also been used in the separation of aromatics containing heteroatoms from hydrocarbon mixtures as disclosed in U.S. Pat. Nos. 5,643,442 and 5,396,019. The aforementioned patents, however, disclose membrane separation processes directed to the general separation of aromatics and non-aromatics using mainly hydrophobic membranes.

The proposed mandate for lowered sulfur levels in gasoline has made it imperative to improve or replace existing methods for desulfurization of petroleum fractions. A more cost-effective method for reducing sulfur content in petroleum fractions is a primary goal of the oil refining industry.

### SUMMARY OF THE INVENTION

This invention relates to the separation of sulfur compounds from liquid hydrocarbon mixtures using a non-ionic membrane. The membrane may be composed of any non-ionic material that preferentially permeates sulfur compounds over hydrocarbons. Preferred membrane materials are hydrophilic. The method of separation includes contacting a liquid hydrocarbon mixture containing sulfur compounds with the membrane and allowing permeation of the liquid hydrocarbon mixture through the membrane creating a sulfur-rich fraction and a sulfur-lean fraction.

The present invention provides a method of separating sulfur compounds from a liquid hydrocarbon mixture using a hydrophilic, non-ionic membrane, said liquid hydrocarbon mixture containing at least one sulfur compound and hydrocarbons, comprising the steps of:

(a) contacting said liquid hydrocarbon mixture with said membrane;

(b) selectively permeating said sulfur compounds of said liquid hydrocarbon mixture through said membrane forming a sulfur-rich permeate and a sulfur-lean retentate; and

(c) retrieving said sulfur-rich permeate and said sulfur-lean retentate.

### BRIEF DESCRIPTION OF THE DRAWINGS

FIG. 1 illustrates the results of pervaporative separation experiments at 115° C. using authentic 180-330° F. gasoline feed and a membrane comprising PVP.

FIG. 2 illustrates the results of pervaporative separation experiments at 115° C. using authentic 180-330° F. gasoline feed and a membrane comprising CTA.

FIG. 3 is a comparative example illustrating the relatively poor separation of sulfur compounds using a polyimide membrane.

FIGS. 4a-4d illustrate the increase in permeation selectivity for thiophene as a function of increasing temperature using a membrane comprising PVP.

FIG. 5 illustrates one embodiment of the process of the present invention for the separation of a hydrocarbon mixture into sulfur-rich and sulfur-lean fractions using a non-ionic membrane.

### DETAILED DESCRIPTION OF PREFERRED EMBODIMENTS

As used herein, “liquid hydrocarbon mixtures” refers to both synthetic mixtures and authentic oil refining fractions, each of which contain sulfur compounds. Preferable liquid hydrocarbon mixtures include FCC gasoline mixtures and light cracked naphthas (LCN). Hydrocarbons in the mixture encompass aliphatic, aromatic, saturated, and unsaturated compounds composed substantially of carbon and hydrogen. Preferable hydrocarbons are compounds that are commonly found in oil refining fractions including, but not limited to, benzene, toluene, naphthenes, olefins, and parrafins. The sulfur compounds in the liquid hydrocarbon mixtures may be in any concentration, but very low levels of from about 1 ppm to about 10,000 ppm are preferred. Also the term, “sulfur compounds” means inorganic or organic compounds comprising at least one sulfur atom. Preferably, sulfur compounds are thiophenes and derivatives thereof.

As used herein, “permeate” refers to the portion of the liquid hydrocarbon mixture that diffuses across a membrane and “retentate” refers to the portion of the liquid hydrocarbon mixture that does not pass through the membrane. Accordingly, the term “permeate side” refers to that side of the membrane on which permeate collects and the term “retentate side” refers to that side of the membrane which contacts the liquid hydrocarbon mixture. Furthermore, the term “sulfur-rich” means having an increased content of sulfur relative to the liquid hydrocarbon mixture, and “sulfur-lean” means having a decreased content of sulfur relative to the liquid hydrocarbon mixture.

As used herein, “hydrophilic” means having an affinity for water or polar compounds. Additionally, “ionic” means having acidic or charged chemical groups and “non-ionic” means having neutral chemical groups.

According to the present invention, “membrane system” is a component of a process that preferentially separates sulfur compounds from liquid hydrocarbon mixtures. The membrane system is single-staged containing one membrane module, or multi-staged containing more than one membrane module. “Membrane module” refers to a membrane assembly comprising a membrane, feed and permeate spacers, and support material, assembled such that there are at least two compartments separated by the membrane. The membrane module may be any workable configuration such as flat sheet, hollow fibers, or spiral-wrapped.

The liquid hydrocarbon mixtures treated by the present invention encompass both synthetic mixtures and authentic oil refining fractions, each of which contain sulfur compounds and hydrocarbons. Preferred liquid hydrocarbon mixtures include FCC gasoline mixtures and light cracked naphtha (LCN). The sulfur compounds in the liquid hydrocarbon mixtures may be in any concentration, but levels of from about 1 ppm to about 10,000 ppm are preferred, and levels of from about 10 ppm to about 4000 ppm are more preferred. The sulfur compounds can also be of any type, including inorganics, however organic compounds are preferred and thiophenes and derivatives thereof are most preferred. Hydrocarbons in the mixture encompass aliphatic, aromatic, saturated, and unsaturated compounds composed essentially of carbon and hydrogen. Preferable hydrocarbons are compounds that are commonly found in oil refining fractions, that are liquid at standard temperature and pressure, including, but not limited to, benzene, toluene, naphthenes, olefins, and paraffins.

According to the methods of the present invention, the membrane separation of sulfur compounds from the liquid hydrocarbon mixtures involves the selective permeation, or diffusion, of sulfur compounds through a membrane. Generally, but not always, selective sorption of components of a mixture is controlled by the affinity of the components for the membrane. Components with greater affinity for the membrane generally permeate more rapidly. Thus, in the present invention, non-ionic membranes which have affinity for, or preferentially permeate, sulfur compounds usually are preferable. Hydrophilic non-ionic membranes are most preferred. One way to define the hydrophilicity of a polymer material is to measure the swelling (percent weight gain) of that polymer in a hydrocarbon solution such as gasoline. Hydrophilic polymers will gain much less weight compared to hydrophobic polymers. Examples of such hydrophilic, non-ionic membrane materials include, but are not limited to, cellulose triacetate (CTA) and polyvinylpyrrolidone (PVP). Hydrophilic properties of the membrane apparently enhance the selectivity of sulfur compounds which are usually more polar than hydrocarbons. Furthermore, the PVP and CTA membranes show a surprising, but desirable, simultaneous increase in flux and selectivity upon increasing temperature of feed. This result is in contrast to what has been observed for hydrophobic membranes, such as polyimides, under similar conditions which usually show a decrease in selectivity and an increase in flux with increasing temperature.

The present invention is related to processes for the separation of sulfur compounds from liquid hydrocarbon mixtures. According to these processes, a liquid hydrocarbon mixture is divided into a sulfur-rich fraction and a sulfur-lean fraction using a membrane system. The sulfur-rich fraction, or sulfur-rich permeate, corresponds to the portion of the liquid hydrocarbon mixture that diffused through the membrane. The sulfur-lean fraction, or sulfur-lean retentate, corresponds to the portion of the liquid hydrocarbon mixture that does not pass through the membrane. Either fraction may be optionally treated again or repeatedly with the separating membrane process for further purification until desired sulfur levels are achieved. Preferably, the sulfur content in the sulfur-lean retentate is from about 1 ppm to about 300 ppm, more preferably about 1 ppm to about 100 ppm, and most preferably from about 1 ppm to about 50 ppm. The sulfur-lean retentate of the membrane separation process would likely be used in fuel formulations, and the sulfur-rich permeate would undergo conventional hydrotreating for further sulfur removal.

The membrane system of the separation process can be single-staged such that it is composed of one membrane module, or may be multi-staged such that it is composed of more than one membrane module. Each module has at least two compartments separated by a layered membrane assembly, the assembly comprising a membrane, feed spacers, and support material. Membrane modules can be any reasonable size and shape, including hollow fibers, stretched flat sheet, or preferably, spiral-wound envelopes. In the spiral-wound configuration, the open sides of membrane envelopes are positioned and sealed over a permeate receptacle such as perforated piping. The envelopes are spirally wrapped around the receptacle to minimize volume. Feed spacers, composed of materials such as plastic netting or nylon mesh, separate the membrane envelopes to allow penetration of the liquid hydrocarbon mixture between the wrapped layers. The interior of each membrane envelope is fitted with a permeate spacer to channel permeate toward the receptacle. The permeate spacer is composed of a material that is flexible, porous, and inert such as polyester. Cushions, composed of a flexible, inert material may flank either side of the permeate spacer inside the membrane envelope and contribute to structural integrity of the membrane assembly under applied pressure.

The membrane possesses certain qualities to function effectively in a process for separating sulfur compounds from liquid hydrocarbon mixtures. In addition to selectivity for sulfur compounds, desirable membrane qualities include resistance to operative conditions such as thermal stress, sustained hydraulic pressure, and prolonged contact with organic chemical mixtures. Membrane thickness may vary from about 0.1 microns to about 200 microns, but thinner membranes are preferred for maximum flux such as, for example, membranes having a thickness of about 0.1 microns to about 50 microns, or more preferably, about 0.1 microns to about 1 micron.

The membranes may take any convenient for known in the art. The preferred form is a composite membrane, that is, a membrane having multiple layers, such as are made by Membrane Technology and Research, Inc. of Menlo Park, Calif. Modem composite membranes typically comprise a highly permeable but relatively non-selective microporous support membrane, which provides mechanical strength, coated with at least one thin selective layer of another material, in the present case the non-ionic, hydrophilic polymer, that is primarily responsible for the separation properties.

Typically, but not necessarily, such a composite membrane is made by solution-casting the support membrane, then solution-coating the selective layer. General preparation techniques for making composite membranes of this type are well known, and are described, for example in U.S. Pat. No. 4,243,701 to Riley et al., incorporated herein by reference, and in U.S. Pat. Nos. 4,931,181 and 4,963,165 to MTR, all of which are incorporated therein by reference.

The microporous support membrane should have a flow resistance that is very small compared to the permselective layer. Preferred support membranes are asymmetric, having a relative open, porous substrate with a thin, dense, finely porous skin layer. The making of such membranes is well known in the art. Preferably the pores in the skin layer should be less than 1 micron in diameter, to enable it to be coated with a defect-free permselective layer. Polymers that may be used to form the microporous support membrane include polysulfone, polimide, polyvinylidene fluoride, polyamide, polypropylene, or polytetrafluoroethylene.

The support membrane is typically reinforced by casting it on a fabric web, made of polyester, for example.

Optionally, the membrane may also include additional layers, such as a gutter layer between the microporous support membrane and the selective layer, or a sealing layer on top of the selective layer.

The membrane system can be operated under either perstraction or pervaporation conditions. Under perstraction conditions, a liquid sweep stream passes across the permeate side of the membrane, dissolving and removing permeated sulfur compounds. In this manner, a concentration gradient is maintained, driving the transfer of sulfur compounds from the retentate side of the membrane to the permeate side. The sweep liquid preferably has affinity for, and is miscible with, the permeated components. Under pervaporation conditions, a vacuum is pulled on the permeate side of the membrane, thus removing permeate as a vapor and sustaining the driving force with a pressure differential. The vapor is cooled and condensed to a liquid and may be optionally heated prior to delivery to subsequent membrane modules. A detailed discussion of perstraction and pervaporation can be found in Membrane Handbook, W. S. Ho and K. K. Sirkar, Eds., Chapman and Hall, 1992, herein incorporated by reference.

Typical process conditions according to the present invention depend on several variables including membrane separation method (i.e., pervaporation vs. perstraction) and feed composition. Determination of appropriate pervaporative and perstractive operating conditions is well within the capabilities of one skilled in the art. Some typical operating parameters for perstractive processes of the present invention include absolute membrane flux of from about 0.5 to about 150 kg·m^{-2·}D⁻¹, feed temperature of from about 20° C. to about 300° C., and negligible pressure drop across the membrane. Additionally, some typical operating parameters for pervaporative processes of the present invention absolute membrane flux of from about 0.5 to about 150 kg·m⁻²·D⁻¹, feed temperature of from about 20° C. to about 300° C., and lowered pressure on the permeate side measuring from about 1 to about 80 mmHg.

Advantages of the present invention are numerous. The separation of sulfur compounds from liquid hydrocarbon mixtures, such as, for example, oil refining fractions, allows the concentration of sulfur contaminants such that a smaller total volume of liquid needs to be processed by conventional hydrotreating. Additionally, selectivity of the membrane for sulfur compounds over unsaturated hydrocarbons results in a low olefin content in the sulfur-rich stream and reduced octane loss and hydrogen consumption during the hydrotreating process.

Those skilled in the art will appreciate that numerous changes and modifications may be made to the preferred embodiments of the present invention, and that such changes and modifications may be made without departing from the spirit of the invention. It is, therefore, intended that the appended claims cover all such equivalent variations as fall within the true spirit and scope of the present invention.

### EXAMPLES

### Example 1

Swelling in Gasoline to Measure the Hydrophlic Nature of a Polymer.

To measure the swelling (weight gain), films of various polymers were prepared and soaked in gasoline over five days at room temperature. Any polymer which gained weight due to gasoline adsorption less than 3% were considered good candidate for membrane separation of sulfur compounds from liquid hydrocarbon mixtures.

TABLE I
Gasoline Composition

| Line # | Components | Wt % |
|---|---|---|
| 1 | n-Paraffins (C₄-C₁₀) | 14.11 |
| 2 | i-Paraffins (C₄-C₁₀) | 23.49 |
| 3 | Olefins (C₄-C₁₀) | 20.99 |
| 4 | Naphthenes (C₅-C₁₀) | 10.69 |
| 5 | Aromatics (C₆-C₁₀) | 30.58 |

TABLE II
Polymer Swelling (Weight Gain) in Gasoline

| Line # | Polymer Films | % Wt. Gain |
|---|---|---|
| 1 | Cellulose Triacetate | 0 |
| 2 | Cellulose | 0 |
| 3 | Polyvinylpyrrolidone | 2.8 |
| 4 | Polyethyleneoxide | 6.2 |
| 5 | Pellethan 2355 (Polyurethane) | 22.2 |
| 6 | Ethyl Cellulose | Dissolved |
| 7 | Polystyrene | Dissolved |

In addition to polymers which had less swelling in gasoline polymer glass transition temperature is also considered. Higher the glass transition temperature better membrane pervaporation performance is expected.

### Example 2

Permeability of Components of a Liquid Hydrocarbon Mixture Using a Membrane Comprising PVP.

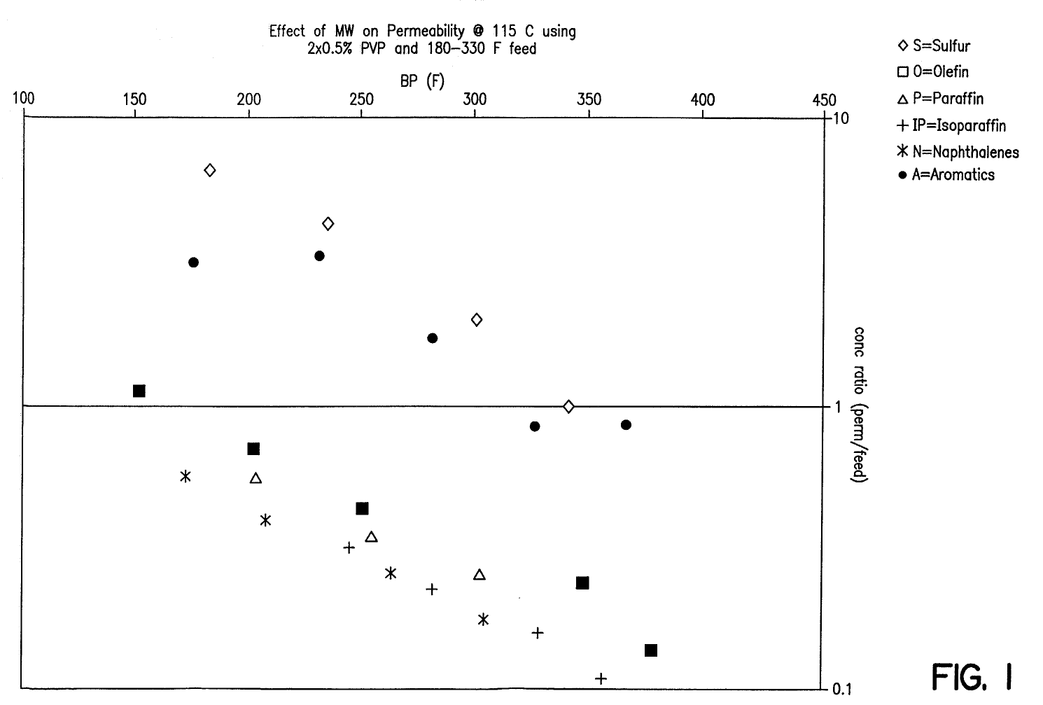

FIG. 1 illustrates the results of pervaporative separation experiments at 115° C. using authentic 180-330° F. gasoline feed and a composite polyvinylpyrrolidone (PVP) membrane. These plots show the selectivity enhancement of the organic sulfur compounds over each of the aromatic and non-aromatic components for the entire molecular weight range. As observed in FIG. 1, the permeation preference for organic compounds is higher than that for, among other compounds, aromatics, olefins and paraffins. Accordingly, efficient separation of sulfur-rich and sulfur-lean fractions was obtained using a PVP membrane.

### Example 3

Permeability of Components of a Liquid Hydrocarbon Mixture Using a Membrane Comprising CTA.

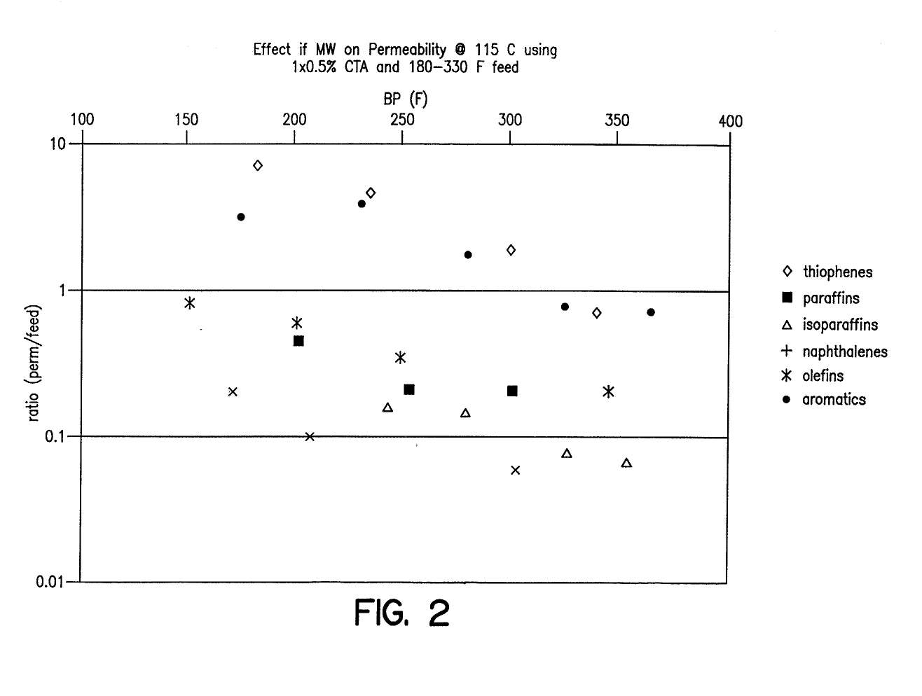

FIG. 2 illustrates the results of pervaporative separation experiments at 115° C. using authentic 180-330° F. gasoline feed and a composite cellulose triacetate (CTA) membrane. These plots show the selectivity enhancement of the organic sulfur compounds over each of the aromatic and non-aromatic components for the entire molecular weight range. As observed in FIG. 2, the permeation preference for organic compounds is higher than that for, among other compounds, aromatics, olefins and paraffins. Accordingly, efficient separation of sulfur-rich and sulfur-lean fractions was obtained using a CTA membrane.

### Example 4

Permeability of Components of a Liquid Hydrocarbon Mixture Using a Hydrophobic Membrane

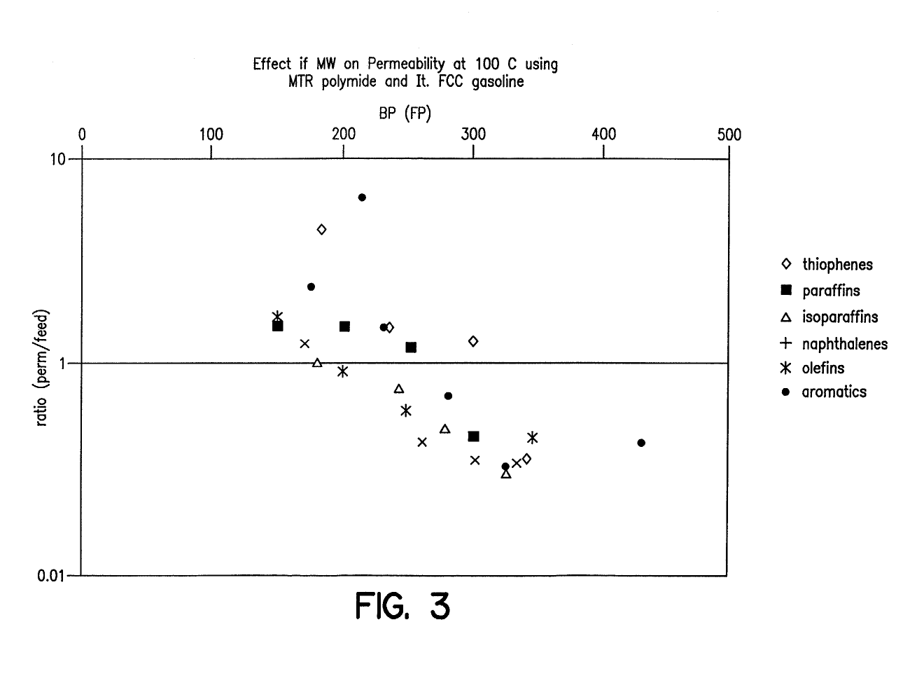

FIG. 3 illustrates the poor separation of sulfur compounds using a hydrophobic polyimide composite membrane. FIG. 3 shows that a hydrophobic membrane such as a polyimide membrane exhibited far less preference for the permeation of the heavier sulfur compounds over the non-sulfur aromatic and non-aromatic compounds.

### Example 5

Temperature Effect on Flux and Selectivity of Membranes Comprising PVP.

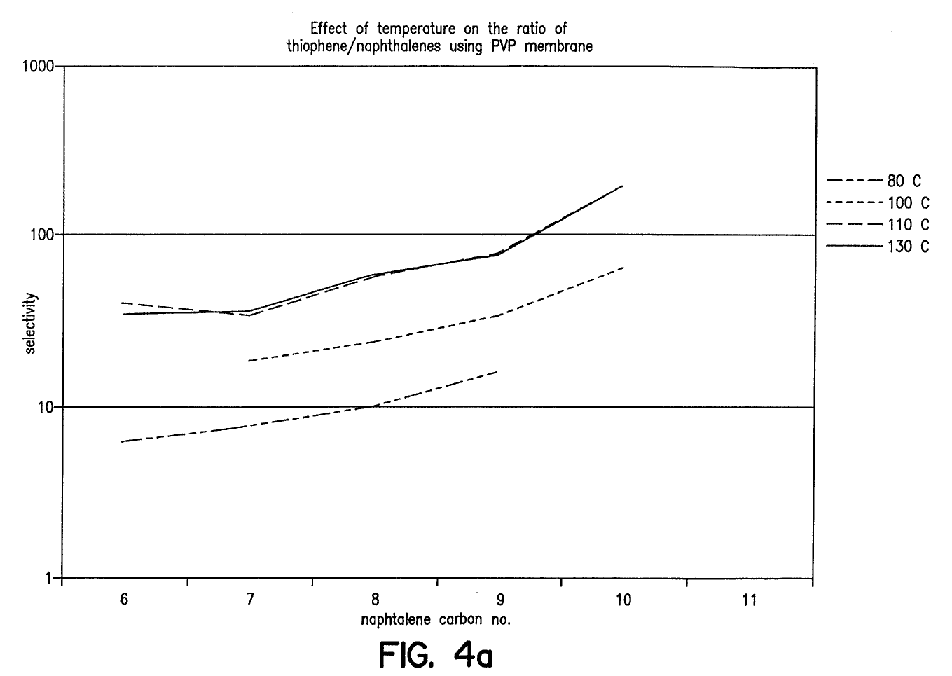

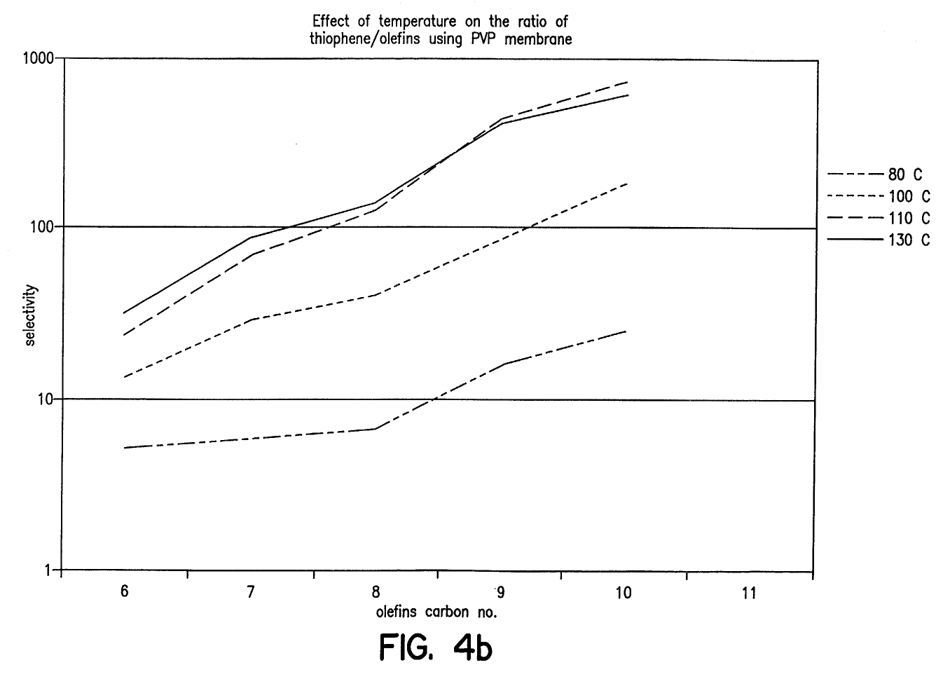

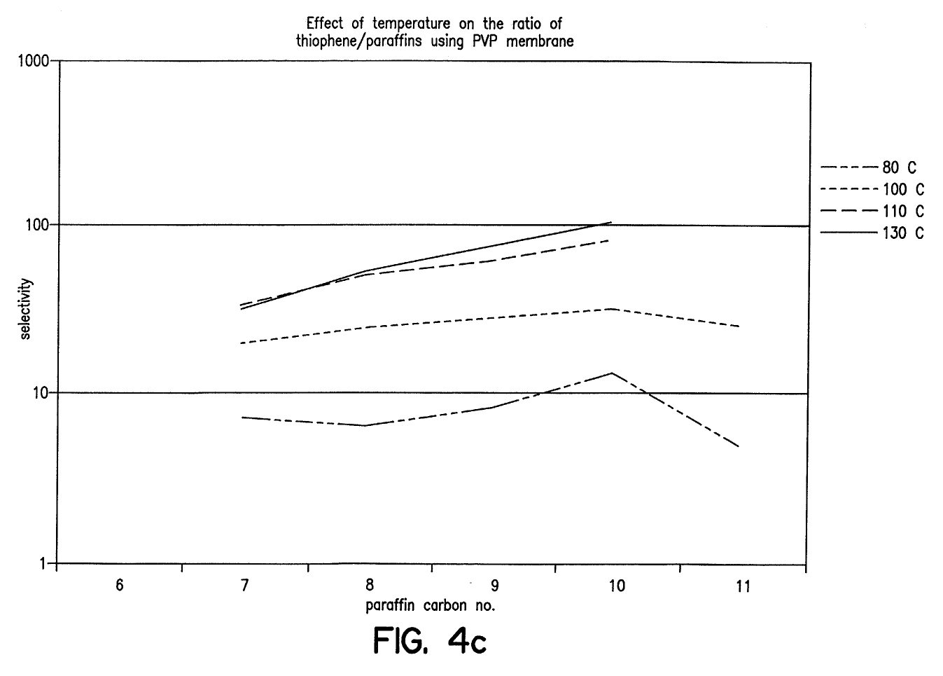

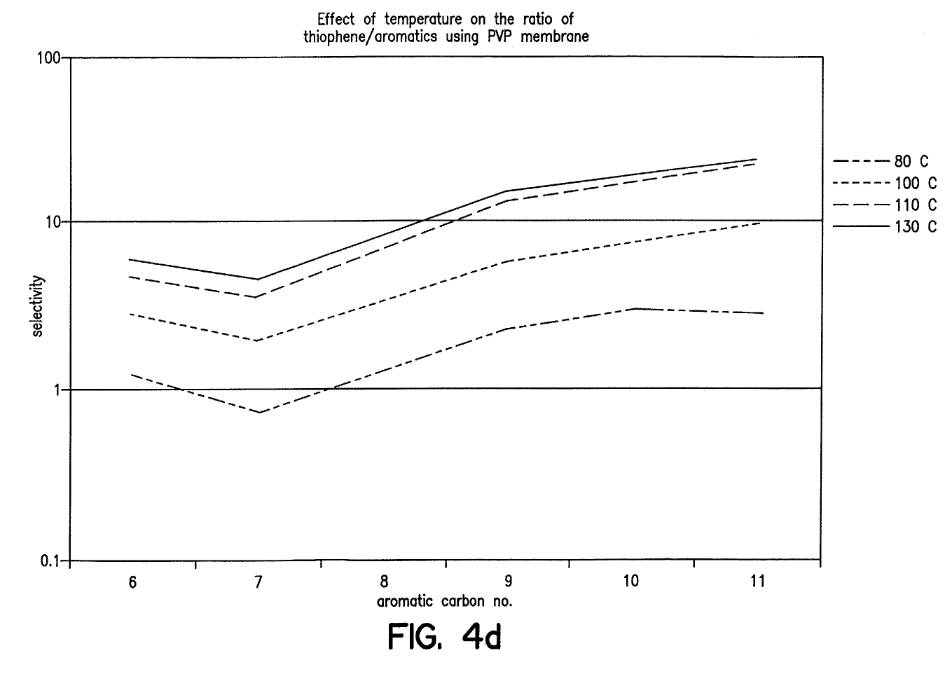

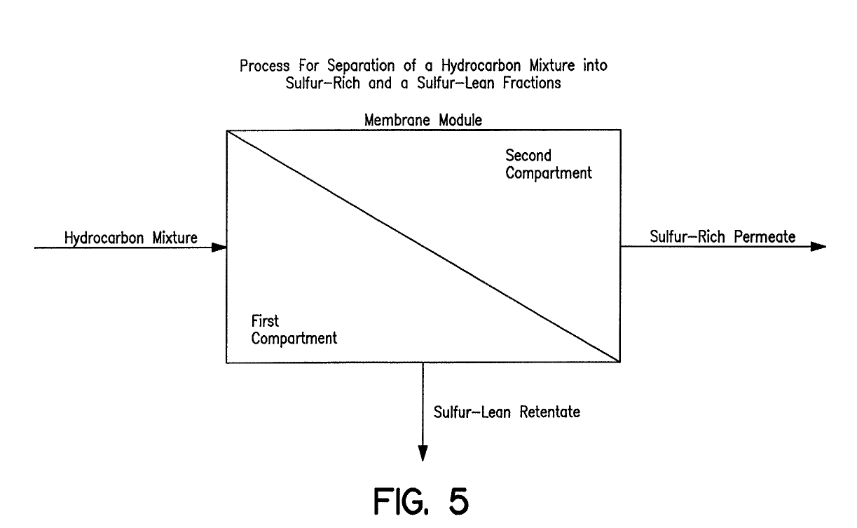

FIGS. 4a-d illustrate the increase in permeation selectivity for thiophene over a range of components, as a function of increasing temperature using a membrane comprising PVP. Selectivities improve upon increasing the temperature from 80° C. to 110° C. Total flux also increases from 0.4 GFD at 80° C. to 1.1 GFD at 130° C. As observed in FIGS. 4a-d, although the membrane selectivity for thiophene over the other components was modest at 80° C., this selectivity improved significantly with increasing temperature. There appeared to be no further selectivity advantage beyond a temperature of 130° C.

What is claimed is:

1. A method f separating sulfur compounds from a liquid hydrocarbon mixture using a hydrophilic, non-ionic membrane, said liquid hydrocarbon mixture containing at least one sulfur compound and hydrocarbons, comprising the steps of:
(a) contacting said liquid hydrocarbon mixture with said membrane;
(b) selectively permeating said sulfur compounds of said liquid hydrocarbon mixture through said membrane forming a sulfur-rich permeate and a sulfur-lean retentate; and
(c) retrieving said sulfur-rich permeate and said sulfur-lean retentate.

2. The method of claim 1 wherein said membrane has less than 3% weight gain in a swelling test in gasoline.

3. The method of claim 1 wherein said membrane is a water-soluble membrane.

4. The method of claim 1 wherein said membrane comprises polyvinylpyrrolidone.

5. The method of claim 1 wherein said non-ionic membrane comprises cellulose triacetate.

6. The method of claim 1 wherein said non-ionic membrane shows a corresponding increase in selectivity with increase in temperature.

7. The method of claim 1 wherein said sulfur compound is thiophene or a derivative of thiophene.

8. The method of claim 1 wherein said sulfur-lean retentate contains from about 1 ppm to about 300 ppm of sulfur.

9. The method of claim 6 wherein said sulfur-lean retentate contains from about 1 ppm to about 100 ppm of sulfur.

10. The method of claim 7 wherein said sulfur-lean retentate contains from about 1 ppm to about 50 ppm of sulfur.

11. The method of claim 1 wherein said liquid hydrocarbon mixture is a light cracked naphtha.

12. The method of claim 1 wherein said liquid hydrocarbon mixture contains from about 10 ppm to about 4000 ppm of sulfur.
